(19) United States
(12) Patent Application Publication
(10) Pub. No.: US 2012/0164633 A1
(43) Pub. Date: Jun. 28, 2012

(54) DIGITAL DROPLET SEQUENCING

(75) Inventor: Thomas Laffler, Vista, CA (US)

(73) Assignee: IBIS BIOSCIENCES, INC., Carlsbad, CA (US)

(21) Appl. No.: 13/337,819

(22) Filed: Dec. 27, 2011

Related U.S. Application Data
(60) Provisional application No. 61/427,291, filed on Dec. 27, 2010.

(51) Int. Cl. C12Q 1/68

(52) U.S. Cl. 435/6.1

(57) ABSTRACT

The present invention provides systems, devices, methods, kits, and compositions for sorting and analysis of nucleic acid sequences using digital droplet PCR. In particular, provided herein are methods to convert complex samples into a plurality of simplified samples, and sequence analysis thereof.

The present Application claims priority to U.S. Provisional Application Ser. No. 61/427,291 filed Dec. 27, 2011, the entirety of which is incorporated by reference herein.

### FIELD OF THE INVENTION

The present invention provides systems, devices, methods, kits, and compositions for sorting and analysis of nucleic acid sequences using digital droplet PCR. In particular, provided herein are methods to convert complex samples into a plurality of simplified samples, and sequence analysis thereof.

### BACKGROUND

Sequencing of mixed nucleic acid samples has typically required a cloning step in order to isolate the different sequences in the mixture. Sequences from a sample are amplified using a set of adapter primers, and the resulting amplicons are placed in vectors and cloned into bacteria to produce isolated clones. Target DNA from the individual clones is then sequenced. Without the cloning step, the sequence data of the mixed sequences is unresolvable due to the complexity from the different sequences in the sample. However, cloning is a time and labor intensive step that greatly slows the process of analyzing nucleic acids from a mixed sample.

### SUMMARY OF THE INVENTION

In some embodiments, the present invention provides methods of analyzing nucleic acid comprising: (a) separating a nucleic acid sample into a plurality of partitions, wherein the nucleic acid sample comprises a mixture of nucleic acid molecules and amplification reagents, wherein a portion of the plurality of partitions are single nucleic acid molecule containing partitions, and a portion of the plurality of partitions are zero nucleic acid molecule containing partitions, and the number of partitions containing more than one nucleic acid molecule is zero, essentially zero, or a statistically insignificant fraction of the total number of partitions; (b) treating the plurality of partitions under amplification conditions such that the single nucleic acid molecule containing partitions become amplicon-containing partitions; and (c) physically sorting the plurality of partitions.

In some embodiments, sorting comprises physically separating the amplicon-containing partitions from partitions not containing amplicons. In some embodiments, sorting comprises physically separating the amplicon-containing partitions from the zero nucleic acid molecule containing partitions. In some embodiments, the each set of primers is configured to amplify a different set of target amplicons. In some embodiments, the different sets of target amplicons are differentially labeled during amplification. In some embodiments, sorting comprises physically separating the differentially labeled sets of target amplicons.

In some embodiments, the present invention further comprises (d) determining the sequence and/or mass of amplicons in the amplicon-containing partitions. In some embodiments, the amplification reagents comprise at least one set of primers. In some embodiments, the amplification reagents comprise two or more sets of primers. In some embodiments, the nucleic acid sample further comprises detection reagents. In some embodiments, the detection reagents comprise labels. In some embodiments, the labels comprise fluorescent labels. In some embodiments, the present invention further comprises a step of labeling the nucleic acid molecules to produce labeled amplicons. In some embodiments, the sample is selected from an environmental sample, a biological sample, a clinical sample, and a forensic sample. In some embodiments, the partitions comprise droplets. In some embodiments, analyzing the sequence or mass of the amplicons comprises determining the nucleotide sequence of all or a portion of the amplicon. In some embodiments, analyzing the mass of the amplicons is performed by mass spectrometry. In some embodiments, the present invention further comprises a step between steps d) amplifying the amplicons to produce clonal populations of amplicons; and e) determining the sequence and/or mass of amplicons in the amplicon-containing partitions.

In some embodiments, the present invention provides systems for performing one or more of the separating, treating, sorting, re-amplifying, and sequencing or mass determination steps of the methods described herein.

In some embodiments, the present invention provides kits of reagents for performing one or more of the separating, treating, sorting, re-amplifying, and sequencing or mass determination steps of the methods described herein. In some embodiments, kits comprise one or more of amplification reagents, detection reagents, sorting reagents, and sequencing reagents.

In some embodiments, the present invention provides methods of analyzing nucleic acid sequences in a sample comprising one or more of the steps of: (a) providing a sample for analysis (e.g. environmental sample, biological sample, clinical sample, forensic sample, etc.), wherein the sample contains or is suspected of containing a mixture of nucleic acid molecules; (b) adding assay reagents to the sample, wherein the assay reagents contain one or more of: buffer, amplification reagents (e.g. primers), detection reagents (e.g. fluorescent labels); (c) partitioning the sample into droplets or other partitions, wherein each droplet contains less than one nucleic acid molecule on average; (d) amplifying the nucleic acid molecules to produce amplicons (e.g. labeled amplicons); (e) detecting the amplicons (e.g. labeled amplicons) or droplets containing amplicons; (f) isolating the droplets containing amplicons (e.g. labeled amplicons); (g) re-amplifying the amplicons to produce clonal populations of amplicons; and (h) analyzing the sequence or mass of the amplicons.

In some embodiments, the present invention provides systems or devices for performing the partitioning, amplification, sorting, and/or sequencing methods described herein.

In some embodiments, the present invention provides kits comprising reagents for performing the one or more of the partitioning, amplification, sorting, and/or sequencing methods described herein. In some embodiments, a kit comprises one or more of amplification reagents, detection reagents, sorting reagents, and sequencing reagents.

### DEFINITIONS

As used herein, the term “partition” refers to a volume of fluid (e.g. liquid or gas) that is a separated portion of a bulk volume. A bulk volume may be partitioned into any suitable number (e.g. 10² . . . 10³ . . . 10⁴ . . . 10⁵ . . . 10⁶ . . . 10⁷, etc.) of smaller volumes (i.e. partitions). Partitions may be separated by a physical barrier or by physical forces (e.g. surface tension, hydrophobic repulsion, etc.). Partitions generated from the larger volume may be substantially uniform in size (monodisperese) or may have non-uniform sizes (polydisperse). Partitions may be produced by any suitable manner (e.g. emulsion, microfluidics, microspray, etc.). Exemplary partitions are droplets.

As used herein, the term “droplet” refers to a small volume of liquid which is immiscible with its surroundings (e.g. gases, liquids, surfaces, etc.). A droplet may reside upon a surface, be encapsulated by a fluid with which it is immiscible (e.g. the continuous phase of an emulsion, a gas (e.g. air, nitrogen)), or a combination thereof. A droplet is typically spherical or substantially spherical in shape, but may be non-spherical. The shape of an otherwise spherical or substantially spherical droplet may be altered by deposition onto a surface. A droplet may be a “simple droplet” or a “compound droplet,” wherein one droplet encapsulates one or more additional smaller droplets. The volume of a droplet and/or the average volume of a set of droplets provided herein is typically less than about one microliter (e.g. 1 μL . . . 0.1 μL . . . 10 pL . . . 1 pL . . . 100 nL . . . 10 nL . . . 1 nL . . . 100 fL . . . 10 fL . . . 1 fL). The diameter of a droplet and/or the average diameter of a set of droplets provided herein is typically less than about one millimeter (e.g. 1 mm . . . 100 μm . . . 10 μm . . . 1 μm). Droplets may be formed by any suitable technique (e.g. emulsification, microfluidics, etc.) and may be monodisperse (e.g., substantially monodisperse) or polydisperse.

As used herein, the term “packet” refers to a set of droplets or other isolated partitions disposed in the same continuous volume, in the same region of a continuous volume, on the same surface, or otherwise grouped. A packet may constitute all of the droplets of bulk volume (e.g. an emulsion), or a segregated fraction of droplets from a bulk volume (e.g. at a range of positions along a channel, containing the same target amplicon, etc.). A packet may constitute all the droplets located along a surface (e.g. chip or microfluidic surface), or the droplets in a defined region of a surface. A packet may refer to a set of droplets that when analyzed in partial or total give a statistically relevant sampling for quantitative analysis of the entire starting sample (e.g. the entire bulk volume).

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the term “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.). Samples may be complex samples or mixed samples, which contain nucleic acids comprising multiple different nucleic acid sequences. Samples may comprise nucleic acids from more than one source (e.g. difference species, different subspecies, etc.), subject, and/or individual. In some embodiments, the methods provided herein comprise purifying the sample or purifying the nucleic acid(s) from the sample. In some embodiments, the sample contains purified nucleic acid. In some embodiments, a sample is derived from a biological, clinical, environmental, research, forensic, or other source.

### DETAILED DESCRIPTION

The present invention provides systems, devices, methods, kits, and compositions for sorting and analysis of nucleic acid sequences using digital droplet PCR. In some embodiments, provided herein are methods to convert complex samples (e.g. containing a plurality of different nucleic acid sequences; a.k.a mixed samples) into a plurality of simpler, more easily analyzed samples (e.g. capable of direct sequencing).

In some embodiments, a sample is analyzed for the presence and/or abundance of a target nucleic acid sequences in a potentially complex sample which may contain many different nucleic acid sequences, each of which may or may not contain the target sequence. In some embodiments, a sample is analyzed to determine the proportion of nucleic acid molecules containing a target sequence of interest. In some embodiments, a complex sample is analyzed to detect the presence and/or measure the abundance or relative abundance or multiple target sequences. In some embodiments, methods provided herein are used to determine what sequences are present in a mixed sample and/or in what relative proportions.

In some embodiments, a sample containing multiple nucleic acid sequences is analyzed by methods described herein. In some embodiments, because of the mixture of DNA sequences contained in the sample, direct sequencing of the nucleic acids in the sample would not allow resolution of the individual sequences contained therein. In some embodiments, the sample is divided into many partitions (e.g. droplets, etc.) using methods described herein (e.g. emulsion, microspray, microfluidics, etc.). In some embodiments, the partitions average less than one nucleic acid molecule per partition. In some embodiments, each partition contains zero or one nucleic acid molecules. In some embodiments, the sample is partitioned in such a manner to reduce the likelihood (e.g., approaching nil) of any partition containing two or more nucleic acid molecules. In some embodiments, the volume and number of partitions is based on the total sample volume and concentration of nucleic acid molecules present in the bulk sample in order to ensure zero or one nucleic acid molecules per partition. In some embodiments, partitioning conditions are optimized to reduce the likelihood of multiple nucleic acid molecules in a single partition.

In some embodiments, the present invention provides amplification (e.g. PCR amplification) of the partitioned nucleic acids of a sample. In some embodiments, amplification reagents (e.g. primers) are added to a sample prior to partitioning and/or concurrent with partitioning, or amplification reagents are added to the partitioned sample. In some embodiments, primers are hybridized to template nucleic acids prior to partitioning. In some embodiments, all partitions are subjected to amplification conditions (e.g. reagents and thermal cycling), but amplification only occurs in partitions containing target nucleic acids (e.g. nucleic acids containing sequences complimentary to primers added to the sample). In some embodiments, amplification of nucleic acids in partitioned samples results in some partitions containing multiple copies of target nucleic acids and other partitions containing no nucleic acids and/or no target nucleic acids (e.g. containing one non-target nucleic acid molecule).

In some embodiments, detection reagents (e.g., fluorescent labels) are included with amplification reagents added to the bulk or partitioned sample. In some embodiments, amplification reagents also serve as detection reagents. In some embodiments, detection reagents are added to partitions following amplification. In some embodiments, detection reagents comprise fluorescent labels. In some embodiments, amplified target nucleic acids (amplicons) are detectable via detection reagents in their partition. In some embodiments, unamplified and/or non-target nucleic acid molecules are not detected. In some embodiments, partitions containing amplified nucleic acids are detectable using one or more detection reagents (e.g. fluorescent labels). In some embodiments, partitions that do not contain amplified nucleic acid, contain unamplified nucleic acid, and/or contain no nucleic acid are either detectable as such, or are undetectable. In some embodiments, measurements of the relative proportion of target nucleic acids in a sample (e.g. relative to other targets nucleic acids, relative to non-target nucleic acids, relative to total nucleic acids, etc.) or the concentration of target nucleic acids in a sample can be measured based on the detection of partitions containing amplified target sequences.

In some embodiments, following amplification, partitions containing amplified target nucleic acids (amplicons) are sorted from partitions not containing amplicons, from partitions not containing nucleic acids, or from amplicons containing other amplified targets. In some embodiments, partitions are sorted based on physical, chemical, and/or optical characteristics of the partition, the nucleic acids therein (e.g. concentration), and/or labels therein (e.g. fluorescent labels). In some embodiments, individual partitions are isolated for subsequent manipulation, processing, and/or analysis of the amplicons therein. In some embodiments, partitions containing similar characteristics (e.g. same fluorescent labels, similar nucleic acid concentrations, etc.) are grouped (e.g. into packets) for subsequent manipulation, processing, and/or analysis (e.g. of the partitions or of the amplicons therein, etc.).

In some embodiments, amplified and/or sorted nucleic acids are re-amplified to increase amplicon concentration within a partition for subsequent manipulation, processing, and/or analysis. In some embodiments, amplified and/or sorted nucleic acids are re-amplified to incorporate sequencing reagents into amplicons. In some embodiments, amplified, sorted, and/or re-amplified target nucleic acid molecules are sequenced according to sequencing methods understood in the art. In some embodiment, amplicons are analyzed using compositions and methods understood in the art (e.g. sequencing, mass spectrometry, spectroscopy, hybridization, etc.).

In some embodiments, the present invention provides methods comprising, but not limited to, one or more of the steps of: (I) partitioning (e.g., droplet generation), (II) amplification (and re-amplification), (III) amplicon detection, (IV) amplicon isolation, and (V) sequencing of a (VI) sample, each of which are addressed below.

### I. Partitioning

In some embodiments, the present invention provides systems, devices, and methods for dividing volumes of fluid and/or reagents into partitions (e.g. droplets). In some embodiments, the present invention utilizes partitioning systems, devices, and/or methods. In some embodiments, exemplary partitioning methods and systems include one or more of emulsification, droplet actuation, microfluidics platforms, continuous-flow microfluidics, reagent immobilization, and combinations thereof.

In some embodiments, partitioning is performed to divide a sample into a sufficient number of partition such that each partition contains one or zero nucleic acid molecules. In some embodiments, partitions are produced at small enough size such that each partition contains one or zero nucleic acid molecules. In some embodiments, the number and size of partitions is based on the concentration and volume of the bulk sample. In some embodiments, the number of nucleic acid molecules to be partitioned is low, relative to the number of partitions. In some embodiments, based on the relatively low number of target nucleic acid molecules compared to partitions, the likelihood of a partition containing 2 or more target nucleic acid molecules is low (e.g. 0.1% . . . 0.01% . . . 0.001% . . . 0.0001% . . . 0.00001% . . . 0.000001). In some embodiments, the number of partitions containing 2 or more nucleic acid molecules is zero. In some embodiments, the number of partitions containing 2 or more nucleic acid molecules is essentially zero, or a statistically insignificant fraction of the totally number of partitions.

In some embodiments, the present invention provides systems, methods, and devices for partitioning a bulk volume into partitions (e.g. droplets) by emulsification (Nakano et al. J Biotechnol 2003; 102:117-124; Margulies et al. Nature 2005; 437:376-380; herein incorporated by reference in their entireties). In some embodiments, the present invention provides systems and methods for generating “water-in-oil” droplets (U.S. Pat. App. No. 20100173394; herein incorporated by reference in its entirety).

In some embodiments, the present invention provides microfluidics systems, methods, and devices for partitioning a bulk volume into partitions (U.S. Pat. App. No. 20100236929; U.S. Pat. App. No. 20100311599; U.S. Pat. App. No. 20100163412; U.S. Pat. No. 7,851,184; herein incorporated by reference in their entireties). In some embodiments, microfluidic systems are configured to generate monodisperse droplets (Kiss et al. Anal Chem. 2008 Dec. 1; 80(23): 8975-8981; herein incorporated by reference in its entirety). In some embodiments, the present invention provides microfluidics systems for manipulating and/or partitioning samples. In some embodiments, a microfluidics system comprises one or more of channels, valves, pumps, etc. (U.S. Pat. No. 7,842,248, herein incorporated by reference in its entirety). In some embodiments, a microfluidics system is a continuous-flow microfluidics system (Kopp et al., Science, vol. 280, pp. 1046-1048, 1998; herein incorporated by reference in its entirety). In some embodiments, microarchitecture of the present invention includes, but is not limited to microchannels, microfluidic plates, fixed microchannels, networks of microchannels, internal pumps; external pumps, valves, centrifugal force elements, etc. In some embodiments, the microarchitecture of the present invention (e.g. droplet microactuator, microfluidics platform, and/or continuous-flow microfluidics) is complemented or supplemented with droplet manipulation techniques, including, but not limited to electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidis platform (e.g. continuous flow components) and such combination approaches involving discrete droplet operations and microfluidics elements are within the scope of the invention.

In some embodiments, the present invention provides a droplet microactuator. In some embodiments, a droplet microactuator is capable of effecting droplet manipulation and/or operations, such as dispensing, splitting, transporting, merging, mixing, agitating, and the like. In some embodiments the invention employs droplet operation structures and techniques described in U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on Jan. 30, 2006; U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000, both to Shenderov et al.; U.S. Patent Publication No. 20060254933, entitled “Device for transporting liquid and system for analyzing” published on Nov. 16, 2006 by Adachi et al., the disclosures of which are incorporated herein by reference in their entireties. Droplet manipulation is, in some embodiments, accomplished using electric field mediated actuation. In such embodiments, electrodes are electronically coupled to a means for controlling electrical connections to the droplet microactuator. An exemplary droplet microactuator includes a substrate including a path and/or array of electrodes. In some embodiments, a droplet microactuator includes two parallel substrates separated by a gap and an array of electrodes on one or both substrates. One or both of the substrates may be a plate.

In some embodiments, nucleic acid targets, primers, and/or probes for use in embodiments of the present invention are immobilized to a surface, for example, a substrate, plate, array, bead, particle, etc. In some embodiments, immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In some embodiments, assay reagents and/or target nucleic acids are immobilized to a surface while retaining the capability to interact and/or react in with other reagents (e.g. reagent dispensed from a microfluidic platform, a droplet microactuator, etc.). In some embodiments, reagents (e.g. target nucleic acids, primers, probes, etc.) are immobilized on a substrate and droplets or partitioned reagents are brought into contact with the immobilized regents. In some embodiments, reagent immobilization is involved in other methods and steps of the present invention (e.g. sequence analysis). Techniques for immobilization of nucleic acids and other reagents to surfcase are well understood by those in the art (See, e.g., U.S. Pat. No. 5,472,881; Taira et al. Biotechnol Bioeng. 2005 Mar. 30; 89(7):835-8); herein incorporated by reference in their entireties).

### II. Amplification

In some embodiments, the present invention provides compositions and method for the amplification of nucleic acids (e.g. DNA, RNA, etc.). In some embodiments, amplification is performed on a bulk sample of nucleic acids. In some embodiments, amplification is performed on a sample that has been divided into partitions (e.g. droplets). In some embodiments, an amplification reaction is carried out within each partition. In some embodiments, a partition contains all the reagents necessary for nucleic acid amplification. In some embodiments, amplification is performed on a single nucleic acid target molecule within a partition. In some embodiments, template nucleic acid is the limiting reagent in a partitioned amplification reaction. In some embodiments, a partition contains one or zero target (e.g. template) nucleic acid molecules. In some embodiments, based on the relatively low number of target nucleic acid molecules compared to partitions, the likelihood of a given partition containing 2 or more target nucleic acid molecules is low (e.g. 0.1% . . . 0.01% . . . 0.001% . . . 0.0001% . . . 0.00001%).

In some embodiments, the present invention provides compositions (e.g. primers, buffers, salts, nucleic acid targets, etc.) and methods for the amplification of nucleic acid (e.g. digital droplet amplification, PCR amplification, partitioned amplification, combinations thereof, etc.). In some embodiments, an amplification reaction is any reaction in which nucleic acid replication occurs repeatedly over time to form multiple copies of at least one segment of a template or target nucleic acid molecule (e.g. DNA, RNA). In some embodiments, amplification generates an exponential or linear increase in the number of copies of the template nucleic acid. Amplifications may produce in excess of a 1,000-fold increase in template copy-number and/or target-detection signal. Exemplary amplification reactions include, but are not limited to the polymerase chain reaction (PCR) or ligase chain reaction (LCR), each of which is driven by thermal cycling. Amplifications used in method or assays of the present invention may be performed in bulk and/or partitioned volumes (e.g. droplets). Alternative amplification reactions, which may be performed isothermally, also find use herein, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and the like.

Amplification may be performed with any suitable reagents (e.g. template nucleic acid (e.g. DNA or RNA), primers, probes, buffers, replication catalyzing enzyme (e.g. DNA polymerase, RNA polymerase), nucleotides, salts (e.g. MgCl₂), etc. In some embodiments, an amplification mixture includes any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), etc.

In some embodiments, the present invention utilizes nucleic acid amplification that relies on alternating cycles of heating and cooling (i.e., thermal cycling) to achieve successive rounds of replication (e.g., PCR). In some embodiments, PCR is used to amplify target nucleic acids (e.g. partitioned targets). PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. Typical PCR methods produce an exponential increase in the amount of a product amplicon over successive cycles, although linear PCR methods also find use in the present invention.

Any suitable PCR methodology, combination of PCR methodologies, or combination of amplification techniques may be utilized in the partitioned methods (e.g. droplet-based detection, separtation, and/or sequencing of target nucleic acids) disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, etc.

In some embodiments, the present invention provides digital PCR methods. In some embodiments, PCR is performed on portions of a sample (e.g. partitions) to determine the presence or absence, concentration, and/or copy number of a nucleic acid target in the sample, based on how many of the sample portions support amplification of the target. In some embodiments, PCR is performed on portions of a sample (e.g. partitions) to detect more than one target nucleic acid and/or to determine the concentration, and/or relative concentrations of multiple target nucleic acids within a sample. In some embodiments, digital PCR is performed as endpoint PCR (e.g., for each of the partitions). In some embodiments, digital PCR is performed as rtPCR (e.g., for each of the partitions).

PCR theoretically results in an exponential amplification of a nucleic acid sequence (e.g. template or target nucleic acid) from a sample. By measuring the number of amplification cycles required to achieve a threshold level of amplification (as in real-time PCR), the starting concentration of nucleic acid can be calculated. However, there are many factors the affect the exponential amplification of the PCR process, such as varying amplification efficiencies, low copy numbers of starting nucleic acid, and competition with background contaminant nucleic acid. Digital PCR is generally insensitive to these factors, since it does not rely on the assumption that the PCR process is exponential. In digital PCR, individual nucleic acid molecules are separated from the initial sample into partitions, and then amplified to detectable levels. Each partition then provides digital information on the presence or absence of each individual nucleic acid molecule within each partition. When enough partitions are measured using this technique, the digital information can be consolidated to make a statistically relevant measure of starting concentration for the nucleic acid target in the sample. In embodiments in which multiple target nucleic acids are analyzed, digital PCR provides statistically relevant measure of the relative concentrations or ratios to multiple target nucleic acids.

In some embodiments, the present invention provides qualitative PCR. In some embodiments, qualitative PCR-based analysis determines whether or not a target is present in a sample (e.g. whether or not a target is present in a partition), generally without any substantial quantification of target. In some embodiments, digital PCR that is qualitative may be performed by determining whether a partition or droplet is positive for the presence of target. In some embodiments, qualitative digital PCR is used to determine the percentage of partitions in a packet that are positive for the presence of target. In some embodiments, qualitative digital PCR is used to determine whether a packet of droplets contains at least a threshold percentage of positive droplets (i.e. a positive sample). In some embodiments, qualitative PCR is performed to detect the presence of multiple targets in a sample.

In some embodiments, the present invention provides RT-PCR (reverse transcription-PCR). In some embodiments, the present invention provides real-time PCR. In some embodiments, the present invention provides endpoint PCR.

### III. Amplicon Detection

In some embodiments, a sample is partitioned using any suitable method, and a nucleic acid amplification procedure is performed to amplify target nucleic acids present in one or more of the partitions. A detection method is then utilized to identify partitions containing amplified target nucleic acids. In some embodiments, the present invention provides systems, devices, methods, and compositions to identify the presence of nucleic acids (e.g. amplicons, labeled nucleic acids) in a sample or partition. In some embodiments, the present invention provides detection of the presence of amplicons in partitions. In some embodiments, the present invention provides detection of partitions in which amplicons were produced. In some embodiments, amplicon detection involves measurement or detection of a characteristic of partitions (e.g. droplets), such as a physical, chemical, luminescence, or electrical aspect, which correlates with amplification (e.g. fluorescence). In some embodiments, the detection method to detect the presence of amplicons within a partition, and/or the identify partitions containing amplification products, is performed by a fluorescence detection technique.

In some embodiments, fluorescence detection methods are provided for detection of amplified nucleic acid, and/or identification of partitions containing amplified nucleic acids. In addition to the reagents already discussed, and those known to those of skill in the art of nucleic acid amplification and detection, various detection reagents, such as fluorescent and non-fluorescent dyes and probes are provided. For example, the protocols may employ reagents suitable for use in a TaqMan reaction, such as a TaqMan probe; reagents suitable for use in a SYBR Green fluorescence detection; reagents suitable for use in a molecular beacon reaction, such as molecular beacon probes; reagents suitable for use in a scorpion reaction, such as a scorpion probe; reagents suitable for use in a fluorescent DNA-binding dye-type reaction, such as a fluorescent probe; and/or reagents for use in a LightUp protocol, such as a LightUp probe. In some embodiments, the present invention provides methods and compositions for detecting and/or quantifying a detectable signal (e.g. fluorescence) from partitions containing amplified target nucleic acid. Thus, for example, methods may employ labeling (e.g. during amplification, post-amplification) amplified nucleic acids with a detectable label, exposing partitions to a light source at a wavelength selected to cause the detectable to fluoresce, and detecting and/or measuring the resulting fluorescence. Fluorescence emitted from the partitions can be tracked during amplification reaction to permit monitoring of the reaction (e.g., using a SYBR Green-type compound), or fluorescence can be measure post-amplification.

In some embodiments, the present invention provides methods of detecting and/or quantifying the presence of a target nucleic acid in partitions by providing a probe with specificity for a target nucleic acid (e.g., a TaqMan-type probe) in partitioned amplification reactions, and detecting the resulting fluorescence. In some embodiments, partitions containing amplified target nucleic acid will exhibit post-amplification fluorescence. In some embodiments, detection of a fluorescent signal is indicative of the presence of the target nucleic acid (e.g. amplified target) in the partition.

The present invention provides corresponding methods for using other suitable target-specific probes (e.g. intercalation dyes, scorpion probes, molecular beacons, etc.), as would be understood by one of skill in the art. In some embodiments, the present invention provides detection of partitions containing amplified nucleic acids and/or the amplicons contained therein, using one or more of fluorescent labeling, fluorescent intercalation dyes, FRET-based detection methods (U.S. Pat. No. 5,945,283; PCT Publication WO 97/22719; both of which are incorporated by reference in their entireties), quantitative PCR, real-time fluorogenic methods (U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995), each of which is incorporated by reference in its entirety), molecular beacons (Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998); herein incorporated by reference in their entiteties), Invader assays (Third Wave Technologies, (Madison, Wis.)) (Neri, B. P., et al., Advances in Nucleic Acid and Protein Analysis 3826:117-125, 2000; herein incorporated by reference in its entirety), nucleic acid sequence-based amplification (NASBA; (See, e.g., Compton, J. Nucleic Acid Sequence-based Amplification, Nature 350: 91-91, 1991; herein incorporated by reference in its entirety), Scorpion probes (Thelwell, et al. Nucleic Acids Research, 28:3752-3761, 2000; herein incorporated by reference in its entirety), capacitive DNA detection (See, e.g., Sohn, et al. (2000) Proc. Natl. Acad. Sci. U.S.A. 97:10687-10690; herein incorporated by reference in its entirety), etc.

### IV. Amplicon Isolation

In some embodiments, the present invention provides methods for sorting and/or isolation of amplified nucleic acid. In some embodiments, the present invention provides methods to sort and/or isolate partitions containing amplified nucleic acid. In some embodiments, following amplification of target sequences and/or detection of amplicons, partitions containing amplicons are sorted for subsequent manipulation (e.g. re-amplification, labeling, restriction digestion, etc.) and/or analysis (e.g. sequencing, mass detection, etc.).

In some embodiments, amplicons are labeled with detectable and/or manipulatable labels (e.g. fluorescent dyes), during or after amplification, by accepted methods understood to those in the art (e.g., intercalation, incorporation, hybridization, etc.) In some embodiments, partitions containing labeled amplicons are detected and/or sorted (e.g. segregated from non-amplicon-containing partitions, grouped according to presence of a particular label, etc.). For example, in some embodiments, amplicon-containing partitions are mechanically separated by micro-manipulators, electrophoresis, flow cytometry, or other sorting techniques known to those in the art. The following references provide guidance for selecting means for analyzing and/or sorting microparticles: Pace, U.S. Pat. No. 4,908,112; Saur et al., U.S. Pat. No. 4,710,472; Senyei et al., U.S. Pat. No. 4,230,685; Wilding et al., U.S. Pat. No. 5,637,469; Penniman et al., U.S. Pat. No. 4,661,225; Kamaukhov et al., U.S. Pat. No. 4,354,114; Abbott et al., U.S. Pat. No. 5,104,791; Gavin et al., PCT publication WO 97/40383; herein incorporated by reference in their entireties.

In some embodiments, partitions containing fluorescently labeled DNA strands are detected, classified, isolated, and/or sorted by fluorescence-activated cell sorting (FACS; See, e.g., Van Dilla et al., Flow Cytometry: Instrumentation and Data Analysis (Academic Press, New York, 1985); Fulwyler et al., U.S. Pat. No. 3,710,933; Gray et al., U.S. Pat. No. 4,361,400; Dolbeare et al., U.S. Pat. No. 4,812,394; herein incorporated by reference in their entireties. In some embodiments, amplcons are fluorescently labeled with detectable and/or manipulatable fluorescent labels, during or after amplification, by accepted methods understood to those in the art (e.g., intercalation, incorporation, hybridization, etc.). In some embodiments, upon excitation with one or more high intensity light sources, such as a laser, a mercury arc lamp, or the like, each partition containing amplified (and labeled) target nucleic acids will generate fluorescent signals. In some embodiments, partitions exhibiting fluorescence above background, or above a threshold level, are sorted by a FACS instrument, according to methods understood by those of skill in the art. Thus, in some embodiments, partitions are sorted according to their relative optical signal, and collected for further analysis by accumulating those partitions generating a signal within a predetermined range of values corresponding to the presence of amplified target nucleic acid. In some embodiments, partitions are sorted and transferred to reaction vessels and/or platforms suitable for subsequent manipulation, processing, and/or analysis.

### V. Sequencing

In some embodiments, the present invention provides compositions and methods for sequencing nucleic acids. In some embodiments, target nucleic acids are sequenced within partitions. In some embodiments, a sample containing nucleic acids is partitioned, target nucleic acid within the partition is amplified, partitions containing amplified nucleic acids are identified and isolated, and amplicons are sequenced. In some embodiments, any suitable systems, devices, compositions, and methods for nucleic acid sequence analysis are within the scope of the present invention. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present invention provides parallel sequencing of partitioned amplcons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

In some embodiments, chain terminator sequencing is utilized. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing in employed (see, e.g., Astier et al., J Am Chem Soc. 2006 Feb 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined.

In certain embodiments, HeliScope by Helicos BioSciences is employed (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Patent Publication No. 20090035777, entitled “HIGH THROUGHPUT NUCLEIC ACID SEQUENCING BY EXPANSION,” that was filed Jun. 19, 2008, which is incorporated herein in its entirety.

Other emerging single molecule sequencing methods include real-time sequencing by synthesis using a VisiGen platform (Voelkerding et al., Clinical Chem., 55: 641-658, 2009; U.S. Pat. No. 7,329,492; U.S. patent application Ser. No. 11/671956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10×10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 liters). At this volume, the activity of a single molecule can be detected amongst a background of thousands of labeled nucleotides.

The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the invention are described in, for example, U.S. Patent No. 7,405,281, entitled “Fluorescent nucleotide analogs and uses therefor”, issued Jul. 29, 2008 to Xu et al., U.S. Pat. No. 7,315,019, entitled “Arrays of optical confinements and uses thereof’, issued Jan. 1, 2008 to Turner et al., U.S. Pat. No. 7,313,308, entitled “Optical analysis of molecules” , issued Dec. 25, 2007 to Turner et al., U.S. Pat. No. 7,302,146, entitled “Apparatus and method for analysis of molecules” , issued Nov. 27, 2007 to Turner et al., and U.S. Pat. No. 7,170,050, entitled “Apparatus and methods for optical analysis of molecules”, issued Jan. 30, 2007 to Turner et al., U.S. Patent Publications Nos. 20080212960, entitled “Methods and systems for simultaneous real-time monitoring of optical signals from multiple sources”, filed Oct. 26, 2007 by Lundquist et al., 20080206764, entitled “Flowcell system for single molecule detection”, filed Oct. 26, 2007 by Williams et al., 20080199932, entitled “Active surface coupled polymerases”, filed Oct. 26, 2007 by Hanzel et al., 20080199874, entitled “CONTROLLABLE STRAND SCISSION OF MINI CIRCLE DNA”, filed Feb. 11, 2008 by Otto et al., 20080176769, entitled “Articles having localized molecules disposed thereon and methods of producing same”, filed Oct. 26, 2007 by Rank et al., 20080176316, entitled “Mitigation of photodamage in analytical reactions”, filed Oct. 31, 2007 by Eid et al., 20080176241, entitled “Mitigation of photodamage in analytical reactions”, filed Oct. 31, 2007 by Eid et al., 20080165346, entitled “Methods and systems for simultaneous real-time monitoring of optical signals from multiple sources”, filed Oct. 26, 2007 by Lundquist et al., 20080160531, entitled “Uniform surfaces for hybrid material substrates and methods for making and using same”, filed Oct. 31, 2007 by Korlach, 20080157005, entitled “Methods and systems for simultaneous real-time monitoring of optical signals from multiple sources”, filed Oct. 26, 2007 by Lundquist et al., 20080153100, entitled “Articles having localized molecules disposed thereon and methods of producing same”, filed Oct. 31, 2007 by Rank et al., 20080153095, entitled “CHARGE SWITCH NUCLEOTIDES”, filed Oct. 26, 2007 by Williams et al., 20080152281, entitled “Substrates, systems and methods for analyzing materials”, filed Oct. 31, 2007 by Lundquist et al., 20080152280, entitled “Substrates, systems and methods for analyzing materials”, filed Oct. 31, 2007 by Lundquist et al., 20080145278, entitled “Uniform surfaces for hybrid material substrates and methods for making and using same”, filed Oct. 31, 2007 by Korlach, 20080128627, entitled “SUBSTRATES, SYSTEMS AND METHODS FOR ANALYZING MATERIALS”, filed Aug. 31, 2007 by Lundquist et al., 20080108082, entitled “Polymerase enzymes and reagents for enhanced nucleic acid sequencing”, filed Oct. 22, 2007 by Rank et al., 20080095488, entitled “SUBSTRATES FOR PERFORMING ANALYTICAL REACTIONS”, filed Jun. 11, 2007 by Foquet et al., 20080080059, entitled “MODULAR OPTICAL COMPONENTS AND SYSTEMS INCORPORATING SAME”, filed Sep. 27, 2007 by Dixon et al., 20080050747, entitled “Articles having localized molecules disposed thereon and methods of producing and using same”, filed Aug. 14, 2007 by Korlach et al., 20080032301, entitled “Articles having localized molecules disposed thereon and methods of producing same”, filed Mar. 29, 2007 by Rank et al., 20080030628, entitled “Methods and systems for simultaneous real-time monitoring of optical signals from multiple sources”, filed Feb. 9, 2007 by Lundquist et al., 20080009007, entitled “CONTROLLED INITIATION OF PRIMER EXTENSION”, filed Jun. 15, 2007 by Lyle et al., 20070238679, entitled “Articles having localized molecules disposed thereon and methods of producing same”, filed Mar. 30, 2006 by Rank et al., 20070231804, entitled “Methods, systems and compositions for monitoring enzyme activity and applications thereof’, filed Mar. 31, 2006 by Korlach et al., 20070206187, entitled “Methods and systems for simultaneous real-time monitoring of optical signals from multiple sources”, filed Feb. 9, 2007 by Lundquist et al., 20070196846, entitled “Polymerases for nucleotide analogue incorporation”, filed Dec. 21, 2006 by Hanzel et al., 20070188750, entitled “Methods and systems for simultaneous real-time monitoring of optical signals from multiple sources”, filed Jul. 7, 2006 by Lundquist et al., 20070161017, entitled “MITIGATION OF PHOTODAMAGE IN ANALYTICAL REACTIONS”, filed Dec. 1, 2006 by Eid et al., 20070141598, entitled “Nucleotide Compositions and Uses Thereof’, filed Nov. 3, 2006 by Turner et al., 20070134128, entitled “Uniform surfaces for hybrid material substrate and methods for making and using same”, filed Nov. 27, 2006 by Korlach, 20070128133, entitled “Mitigation of photodamage in analytical reactions”, filed Dec. 2, 2005 by Eid et al., 20070077564, entitled “Reactive surfaces, substrates and methods of producing same”, filed Sep. 30, 2005 by Roitman et al., 20070072196, entitled “Fluorescent nucleotide analogs and uses therefore”, filed Sep. 29, 2005 by Xu et al., and 20070036511, entitled “Methods and systems for monitoring multiple optical signals from a single source”, filed Aug. 11, 2005 by Lundquist et al., and Korlach et al. (2008) “Selective aluminum passivation for targeted immobilization of single DNA polymerase molecules in zero-mode waveguide nanostructures” Proc. Nat'l. Acad. Sci. U.S.A. 105(4): 11761181—all of which are herein incorporated by reference in their entireties.

### VI. Samples

The amplification methods, compositions, systems, and devices of the present invention make use of samples which include a nucleic acid template. Samples may be derived from any suitable source, and for purposes related to any field, including but not limited to diagnostics, research, forensics, epidemiology, pathology, archaeology, etc. A sample may be biological, environmental, forensic, veterinary, clinical, etc. in origin. Samples may include nucleic acid derived from any suitable source, including eukaryotes, prokaryotes (e.g. infectious bacteria), mammals, humans, non-human primates, canines, felines, bovines, equines, porcines, mice, viruses, etc. Samples may contain, e.g., whole organisms, organs, tissues, cells, organelles (e.g., chloroplasts, mitochondria), synthetic nucleic acid, cell lysate, etc. Nucleic acid present in a sample (e.g. target nucleic acid, template nucleic acid, non-target nucleic acid, contaminant nucleic acid may be of any type, e.g., genomic DNA, RNA, plasmids, bacteriophages, synthetic origin, natural origin, and/or artificial sequences (non-naturally occurring), synthetically-produced but naturally occurring sequences, etc. Biological specimens may, for example, include whole blood, lymphatic fluid, serum, plasma, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs or washes (e.g., oral, nasopharangeal, optic, rectal, intestinal, vaginal, epidermal, etc.) and/or other biological specimens.

In some embodiments, samples that find use with the present invention are mixed samples (e.g. containing mixed nucleic acid populations). In some embodiments, samples analyzed by methods herein contain, or may contain, a plurality of different nucleic acid sequences. In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that contain a target sequence of interest in a particular application. In some embodiments, a sample (e.g. mixed sample) contains zero nucleic acid molecules that contain a target sequence of interest in a particular application. In some embodiments, a sample (e.g. mixed sample) contains nucleic acid molecules with a plurality of different sequences that all contain a target sequence of interest. In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that do not contain a target sequence of interest in a particular application. In some embodiments, a sample (e.g. mixed sample) contains zero nucleic acid molecules that do not contain a target sequence of interest in a particular application. In some embodiments, a sample (e.g. mixed sample) contains nucleic acid molecules with a plurality of different sequences that do not contain a target sequence of interest. In some embodiments, a sample contains more nucleic acid molecules that do not contain a target sequence than nucleic acid molecules that do contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains more nucleic acid molecules that do contain a target sequence than nucleic acid molecules that do not contain a target sequence (e.g. 1.01:1 . . . 2:1 . . . 5:1 . . . 10:1 . . . 20:1 . . . 50:1 . . . 10²:1 . . . 10³:1 . . . 10⁴:1 . . . 10⁵:1 . . . 10⁶:1 . . . 10⁷:1). In some embodiments, a sample contains a single target sequence which may be present in one or more nucleic acid molecules in the sample. In some embodiments, a sample contains a two or more target sequences (e.g. 2, 3, 4, 5 . . . 10 . . . 20 . . . 50 . . . 100, etc.) which may each be present in one or more nucleic acid molecules in the sample.

In some embodiments, various sample processing steps may be accomplished to prepare the nucleic acid molecules within a sample, including, but not limited to cell lysis, restriction digestion, purification, precipitation, resuspension (e.g. in amplification buffer), dialysis, etc. In some embodiments, sample processing is performed before or after any of the steps of the present invention including, but not limited to partitioning, amplification, re-amplification), amplicon detection, amplicon isolation, sequencing, etc.

Although the invention has been described in connection with specific preferred embodiments, it should be understood that the invention as claimed should not be unduly limited to such specific embodiments. Various modification and variation of the described methods and compositions of the invention will be apparent to those skilled in the art without departing from the scope and spirit of the invention. Indeed, various modifications of the described modes for carrying out the invention understood by those skilled in the relevant fields are intended to be within the scope of the following claims. All publications and patents mentioned in the present application and/or listed below are herein incorporated by reference.

We claim:

1. A method of analyzing nucleic acid comprising:
a) separating a nucleic acid sample into a plurality of partitions, wherein said nucleic acid sample comprises a mixture of nucleic acid molecules and amplification reagents, wherein a portion of said plurality of partitions are single nucleic acid molecule containing partitions, and a portion of said plurality of partitions are zero nucleic acid molecule containing partitions, and the number of partitions containing more than one nucleic acid molecule is zero or a statistically insignificant fraction of the total number of partitions;
b) treating said plurality of partitions under amplification conditions such that said single nucleic acid molecule containing partitions become amplicon-containing partitions; and
c) physically sorting said plurality of partitions.

2. The method of claim 1, wherein said sorting comprises physically separating said amplicon-containing partitions from partitions not containing amplicons.

3. The method of claim 1, wherein said sorting comprises physically separating said amplicon-containing partitions from said zero nucleic acid molecule containing partitions.

4. The method of claim 1, wherein said amplification reagents comprise at least one set of primers.

5. The method of claim 4, wherein said amplification reagents comprise two or more sets of primers.

6. The method of claim 5, wherein each set of primers is configured to amplify a different set of target amplicons.

7. The method of claim 6, wherein said different sets of target amplicons are differentially labeled during amplification.

8. The method of claim 7, wherein said sorting comprises physically separating said differentially labeled sets of target amplicons.

9. The method of claim 1, further comprising d) determining the sequence and/or mass of amplicons in said amplicon-containing partitions.

10. The method of claim 1, wherein said nucleic acid sample further comprises detection reagents.

11. The method of claim 10, wherein said detection reagents comprise labels.

12. The method of claim 11, wherein said labels comprise fluorescent labels.

13. The method of claim 11, further comprising a step of labeling the nucleic acid molecules to produce labeled amplicons.

14. The method of claim 1, wherein said sample is selected from an environmental sample, a biological sample, a clinical sample, and a forensic sample.

15. The method of claim 1, wherein said partitions comprise droplets.

16. The method of claim 1, wherein analyzing the sequence or mass of the amplicons comprises determining the nucleotide sequence of all or a portion of the amplicon.

17. The method of claim 1, wherein analyzing the mass of the amplicons is performed by mass spectrometry.

18. The method of claim 1, further comprising the steps of: d) re-amplifying the amplicons to produce clonal populations of amplicons; and e) determining the sequence and/or mass of amplicons in said amplicon-containing partitions.
